(19) United States
(12) Patent Application Publication
(10) Pub. No.: US 2023/0390943 A1
(43) Pub. Date: Dec. 7, 2023

(54) OPENING APPARATUS FOR CONTAINER, ANALYSIS DEVICE, CLOSING APPARATUS FOR CONTAINER, AND REAGENT KIT

(71) Applicant: SHIMADZU CORPORATION, Kyoto (JP)

(72) Inventors: Tomoki SASAYAMA, Kyoto-shi, Kyoto (JP); Nobuhiro HANAFUSA, Kyoto-shi, Kyoto (JP)

(73) Assignee: SHIMADZU CORPORATION, Kyoto (JP)

(21) Appl. No.: 18/033,801

(22) PCT Filed: Sep. 7, 2021
(86) PCT No.: PCT/JP2021/032751

§ 371 Date: Apr. 26, 2023

(30) Foreign Application Priority Data
Oct. 26, 2020 (JP) 2020-179181
Dec. 8, 2020 (JP) 2020-203701

(51) Int. Cl. B25J 15/00; G01N 35/00; B67B 7/16; C12M 1/34; C12N 15/09; C12Q 1/686

(52) U.S. Cl. CPC B25J 15/0033; G01N 35/0099; B67B 7/16; C12M 1/34; C12N 15/09; C12Q 1/686

(57) ABSTRACT

An opening apparatus includes a temperature adjustment section that holds a container body of a PCR container, an opening/closing unit, a moving device, and a control device. The opening and closing unit includes: a rotating member that applies a force to a lid in a direction to open the lid as the rotating member rotates with a projection in contact with the lid in a closed state; and a motor that rotates the rotating member. The moving device moves the temperature adjustment section relative to the rotating member. In conjunction with driving and thus rotating the rotating member, the control device controls the moving device so that the PCR container moves horizontally in a direction from a hinge of the PCR container toward a flange of the PCR container.

### TECHNICAL FIELD

The present disclosure relates to an opening apparatus for a container for receiving an analyte to be analyzed (a biological sample such as blood, urine, bodily fluid obtained with a nasopharyngeal swab, saliva, etc.) or a reagent, an analysis device for analyzing infectious viruses or genes contained in the analyte in the container, and a reagent kit usable in the analysis device.

### BACKGROUND ART

Conventionally, there is an apparatus for analyzing a gene contained in an analyte through polymerase chain reaction (PCR) (for example, see Japanese Patent No. 4785862).

### CITATION LIST

### Patent Literature

PTL 1: Japanese Patent 4785862

### SUMMARY OF INVENTION

### Technical Problem

In order to analyze genes through PCR, it is necessary for an operator who performs analysis to manually perform some of operations such as a reagent adjustment operation prior to PCR and an opening operation for a container for dispensing an analyte or a reagent into the container, and accordingly, the operator needs to be skilled. These operations are cumbersome, and the analysis takes a long time of about several hours, and there is a possibility that the operator may introduce variation into data, make mistakes, be infected, etc. Therefore, gene analysis through PCR is difficult to perform in medical institutions such as small- and medium-sized hospitals and clinics, and is mainly performed in large hospitals, examination centers, and hygiene laboratories. Therefore, medical institutions such as small- and medium-sized hospitals and clinics collect and subsequently transport analytes to facilities that can measure the analytes, and thereafter obtain result data, which takes several days, and it is thus difficult to perform quick measurement.

While Japanese Patent No. 4785862 discloses an apparatus to detect a gene fluorescently or the like while subjecting an analyte to PCR with a thermal cycler, as has been discussed above, dispensing an analyte or a reagent into a container is often performed by a manual operation performed by an operator. Furthermore, while there exists an automated all-in-one apparatus including these, conventional all-in-one apparatuses have a problem in that they use a dedicated container for receiving a reagent and cannot use a commercially available container (a PCR tube), and are thus low in versatility.

The present disclosure has been made in order to solve the above problem, and an object of the present disclosure is to appropriately open a general-purpose commercially available container without depending on an operator's manual operation.

Another object of the present disclosure is to efficiently perform an operation of setting a reagent container for an analysis device.

Another object of the present disclosure is to appropriately close a general-purpose commercially available container without depending on the operator's manual operation.

### Solution to Problem

An opening apparatus according to an aspect of the present disclosure is an opening apparatus for a container including a container body, a lid, and a hinge that interconnects the container body and the lid. The opening apparatus includes: a holding device that holds the container body; an opening device including a rotating member and a drive device that rotates the rotating member, the rotating member having a first projection and a second projection capable of coming into contact with the lid, the rotating member being configured to apply a force to the lid in a direction to open the lid by rotating while one of the first and second projections is in contact with the lid in a closed state; a moving device that moves at least one of the holding device and the rotating member relatively to the other; and a control device that controls the opening device and the moving device in conjunction with each other to open the lid.

According to the opening apparatus, when the rotating member rotates while at least one of the first and second projections is in contact with the lid of the container, a force is applied to the lid in a direction to open the lid. Further, in conjunction with rotation of the rotating member, the holding device holding the container body is relatively moved with respect to the rotating member. Accordingly, the container can be displaced in accordance with positional displacement of the first and second projections with the rotation of the rotating member. Therefore, even if the first and second projections are positionally displaced as the rotating member rotates, the first and second projections no longer coming into contact with the lid can be suppressed. As a result, a general-purpose commercially available container can be appropriately opened without depending on an operator's manual operation.

An analysis device according to an aspect of the present disclosure includes the above-described opening apparatus and a dispensing device that dispenses a reagent into a container opened by the opening apparatus.

According to the analysis device described above, a container can be opened and a reagent can be dispensed into the opened container without depending on an operator's manual operation.

A closing apparatus according to an aspect of the present disclosure is a closing apparatus for a container including a container body, a lid, and a hinge that interconnects the container body and the lid. The closing apparatus includes: a holding device that holds the container body; a closing device including a rotating member and a drive device that rotates the rotating member, the rotating member being configured to apply a force to the lid in a direction to close the lid by rotating in contact with the lid in an open state; and a control device that controls the closing device. When the lid in the open state is closed, the control device performs a first closing control to apply a force to the lid in a direction to close the lid by rotating the rotating member so that a portion of the rotating member approaches the hinge while the portion of the rotating member is in contact with the lid.

According to the above-described closing apparatus, the first closing control pushes the lid toward the hinge, rather than toward a tip of the lid, and thus suppresses positional displacement of the lid with respect to the container body toward the tip of the lid. As a result, there is no interference caused between the lid and the container body, and the lid can be appropriately closed.

A reagent kit according to an aspect of the present disclosure includes a plurality of containers and a packaging material that packages the plurality of containers. Each of the plurality of containers includes a container body and a lid connected to the container body via a hinge and capable of opening and closing with respect to the container body. The plurality of containers are provided such that they are one-dimensionally disposed, with adjacent containers connected to each other, the lid closed, and a reagent introduced in each container body.

According to the reagent kit described above, a reagent container setting operation can be completed simply by setting a plurality of one-dimensionally connected containers in a holding device, rather than setting a plurality of mutually separated containers each in the holding device. As a result, reagent containers can be set efficiently in an analysis device.

### Advantageous Effects of Invention

In the opening apparatus according to the present disclosure, a general-purpose commercially available container can be appropriately opened without depending on an operator's manual operation.

In the closing apparatus according to the present disclosure, a general-purpose commercially available container can be appropriately closed without depending on an operator's manual operation.

In the reagent kit according to the present disclosure, reagent containers can be set efficiently in an analysis device.

### BRIEF DESCRIPTION OF DRAWINGS

FIG. 1 schematically shows an example of a configuration of an analysis system.

FIG. 2 shows a holding device in which a container is set, as viewed in a direction along a Z-axis.

FIG. 3 is a cross section of the holding device in which a container is set, as viewed in a direction along the Y-axis.

FIG. 4 shows a state in which a long pipette tip is attached to a nozzle of a syringe and the long pipette tip is inserted into an analyte container.

FIG. 5 shows a state in which a short pipette tip is attached to a nozzle of a syringe and the short pipette tip is inserted into a reagent container.

FIG. 6 schematically shows each step of an analysis process by an analysis device.

FIG. 7 is a perspective view of an opening/closing mechanism provided in an opening/closing unit.

FIG. 8 schematically shows an operation of a rotating member and a container when an opening operation for a PCR container is performed.

FIG. 9 is a flowchart of an example of a processing procedure when a control device performs an opening operation for a container.

FIG. 10 is a flowchart of an example of a processing procedure when the control device performs a closing operation for a container.

FIG. 11 shows an example of a configuration of an irradiation unit.

FIG. 12 shows an exemplary variation of the configuration of the irradiation unit.

FIG. 13 shows a variation of a configuration of the opening/closing unit.

FIG. 14 shows a state in which a projection of the rotating member is set to a lid of a container in the opening operation for a PCR container.

FIG. 15 shows a state in which the lid of the container is closed by using the projection of the rotating member.

FIG. 16 shows a state in which the lid of the container is closed by using the projection of the rotating member.

FIG. 17 shows an appearance of a commercially available PCR tube.

FIG. 18 schematically shows an appearance of a reagent kit.

FIG. 19 schematically shows a state of the rotating member and PCR container before heating when the opening operation ends.

FIG. 20 schematically shows a state of the rotating member and PCR container after heating when the opening operation ends.

FIG. 21 schematically shows a movement of a short pipette tip with respect to a PCR container.

FIG. 22 schematically shows a closing operation according to comparative example 1.

FIG. 23 schematically shows a closing operation according to comparative example 2.

FIG. 24 schematically shows an operation by a first closing control.

FIG. 25 schematically shows an operation by a second closing control.

### DESCRIPTION OF EMBODIMENTS

Hereinafter, embodiments of the present disclosure will be described in detail with reference to the accompanying drawings. In the figures, identical or corresponding components are identically denoted and will not be described repeatedly.

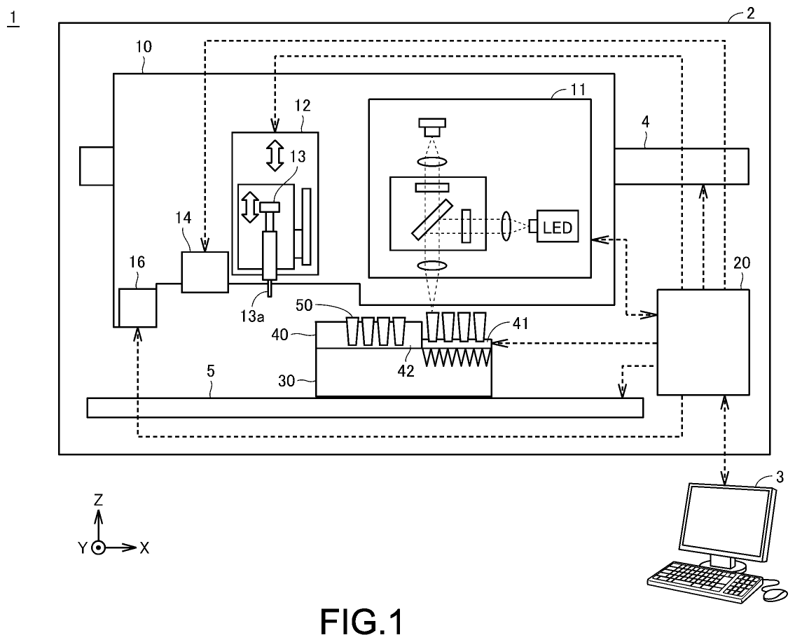

FIG. 1 schematically shows an example of a configuration of an analysis system 1 according to the present embodiment. Analysis system 1 is an apparatus capable of fully automatically performing a process of measuring and analyzing gene amplification by PCR over time (in real time). Hereinafter, as shown in FIG. 1, a direction along a vertical direction (in FIG. 1, an upward/downward direction) will also be referred to as a “Z-axis direction,” and directions perpendicular to the vertical direction and orthogonal to each other will also be referred to as an “X-axis direction” and a “Y-axis direction,” respectively.

Analysis system 1 comprises an analysis device 2 and a terminal 3 capable of communicating with analysis device 2. Terminal 3 is a general personal computer including a display and operated by an operator.

Analysis device 2 includes an inspection device 10, a control device 20, a temperature adjustment device 30, and moving devices 4 and 5. Temperature adjustment device 30 includes a holding device (a holder) 40 configured to be capable of holding a plurality of containers 50 or the like. Holding device 40 includes a temperature adjustment section 41 having a temperature adjustment function (a heating function and a cooling function) by a Peltier element or the like, and a holding section 42 having no temperature adjustment function.

Moving device 4 includes an actuator (not shown) that moves inspection device 10 in a horizontal direction (the X- and Y-axis directions). Moving device 5 includes an actuator (not shown) that moves holding device 40 in the horizontal direction (the X- and Y-axis directions). The actuators of the moving devices 4 and 5 operate in response to a command issued from control device 20. By moving at least one of inspection device 10 and holding device 40 in the horizontal direction by moving devices 4 and 5, a relative distance between inspection device 10 and holding device 40 in the horizontal direction can be adjusted. Either one of moving devices 4 and 5 may be dispensed with.

Inspection device 10 includes an optical unit 11, a dispensing unit 12, an opening/closing unit 14, and an irradiation unit 16.

Dispensing unit 12 is provided with a syringe 13 having a tip to which a nozzle 13a extending in the Z-axis direction is attached. A plunger (not shown) movable in the Z-axis direction is provided inside nozzle 13a. Syringe 13 is configured to suck liquid in an amount corresponding to an amount of a stroke of the plunger in a positive direction along the Z-axis and discharge liquid in an amount corresponding to an amount of a stroke of the plunger in a negative direction along the Z-axis. Dispensing unit 12 includes an actuator (not shown) for moving syringe 13 in the Z-axis direction and an actuator (not shown) for stroking the plunger in nozzle 13a in the Z-axis direction. These actuators operate in response to a command issued from control device 20.

Opening/closing unit 14 includes an opening/closing mechanism having a projection that comes into contact with and automatically opens/closes a lid of container 50 held by holding device 40. Opening/closing unit 14 operates in response to a command issued from control device 20. A configuration of opening/closing unit 14 will be described later in detail.

Irradiation unit 16 prevents contamination by irradiating the projection of opening/closing unit 14 and a vicinity thereof with UV light (a ultraviolet ray) in view of a possibility that when opening/closing unit 14 opens/closes the lid of container 50 an analyte may adhere to the projection of opening/closing unit 14 and contaminate a next analyte. A configuration of irradiation unit 16 will also be described in detail later.

Optical unit 11 is a device for analyzing infectious viruses or genes contained in an analyte in container 50 by detecting fluorescence emitted from the analyte when the analyte is irradiated with excitation light. Optical unit 11 detects fluorescence for three wavelengths of red (R), green (G), and blue (B), respectively, and outputs a result thereof to control device 20. Optical unit 11 includes a light source (e.g., a light emitting diode), a lens for irradiating an analyte with light from the light source and collecting fluorescence of the analyte, etc., and a photodiode for detecting fluorescence emitted from the analyte and converting the detected fluorescence into analyzable digital data, etc. Optical unit 11 may adopt a well-known configuration.

Although not shown, control device 20 includes a CPU (a central processing unit), a memory, an input/output buffer, etc. Upon receiving a start analysis command from terminal 3, control device 20 controls each part of analysis device 2 (each unit in inspection device 10, moving devices 4 and 5, and temperature adjustment section 41 of temperature adjustment device 30) in accordance with a predetermined procedure to analyze infectious viruses or genes contained in the analyte. Control device 20 causes a display of terminal 3 to display an analysis result of analysis device 2.

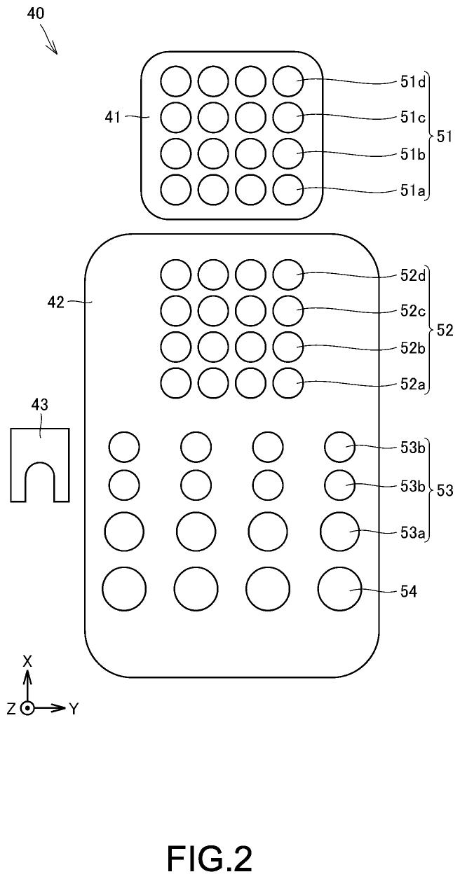

FIG. 2 shows holding device 40 in which container 50 is set, as viewed in a direction along the Z-axis. Holding device 40 has an arrangement surface extending in the XY plane and allowing a plurality of containers 50 to be two-dimensionally disposed therein. Inspection device 10 and holding device 40 are configured to be movable relative to each other two-dimensionally along the arrangement surface of the holding devices 40 by moving devices 4 and

Container 50 disposed in the arrangement surface of holding device 40 includes a PCR container (a reaction container) 51 receiving a liquid to be subjected to a thermal cycle (an analyte to which each reagent is added), a reagent container 52 receiving each reagent, and an analyte container 54 receiving an analyte alone.

Four PCR containers 51a, 51b, 51c and 51d disposed one-dimensionally in the X-axis direction are set as one set, and four such sets of PCR containers 51 are disposed in the Y-axis direction.

Four reagent containers 52a, 52b, 52c and 52d disposed one-dimensionally in the X-axis direction are set as one set, and four such sets of reagent containers 52 are disposed in the Y-axis direction. Reagent container 52a contains an analyte treatment liquid in advance. Reagent container 52b contains a reaction liquid in advance. Reagent container 52c contains a primer/probe liquid (a liquid containing a primer and a probe) in advance. Reagent container 52d contains an enzyme liquid in advance. Four reagent containers 52a, 52b, 52c and 52d are provided (or commercially available) as one set as a reagent kit such that the reagents are sealed in advance in an amount necessary for analyzing at least one analyte. A manner of providing the reagent kit will be described in detail later.

Four analyte containers 54 are disposed one-dimensionally in the Y-axis direction. In analysis device 2 according to the present embodiment, by introducing different analytes in four analyte containers 54, respectively, disposed in the Y-axis direction, four analytes can be analyzed at one time.

A step (a hole or a depression) in which a portion of each container 50 (PCR container 51, reagent container 52, and analyte container 54) can be inserted in the Z-axis direction is formed in holding device 40 at a position where each container 50 is disposed. By inserting each container 50 along a corresponding step, each container 50 is positionally fixed in the X-axis direction and the Y-axis direction.

Furthermore, a dispensing pipette tip 53 for dispensing an analyte and a reagent is also disposed in an area of holding device 40 between reagent container 52 and analyte container 54. Dispensing pipette tip 53 is attached to nozzle 13a of syringe 13 and thus used.

In the present embodiment, dispensing pipette tip 53 includes a long pipette tip 53a used for analyte container 54 and a short pipette tip 53b used for PCR container 51 and reagent container 52 (a pipette tip for a small amount). One long pipette tip 53a and two short pipette tips 53b disposed one-dimensionally in the X-axis direction are provided as one set, and four such sets of dispensing pipette tips 53 are disposed in the Y-axis direction.

PCR container 51 to be subjected to a thermal cycle is disposed in temperature adjustment section 41 having a temperature adjustment function, and reagent container 52, dispensing pipette tip 53 and analyte container 54 are disposed in holding section 42 having no temperature adjustment function.

Further, holding device 40 is provided with a pipette tip discarding section 43 for discarding dispensing pipette tip 53 used.

Although not shown in FIG. 2, each container 50 is a resin molded product having a lid and a container body integrated together, and is configured to allow the lid to be opened/closed.

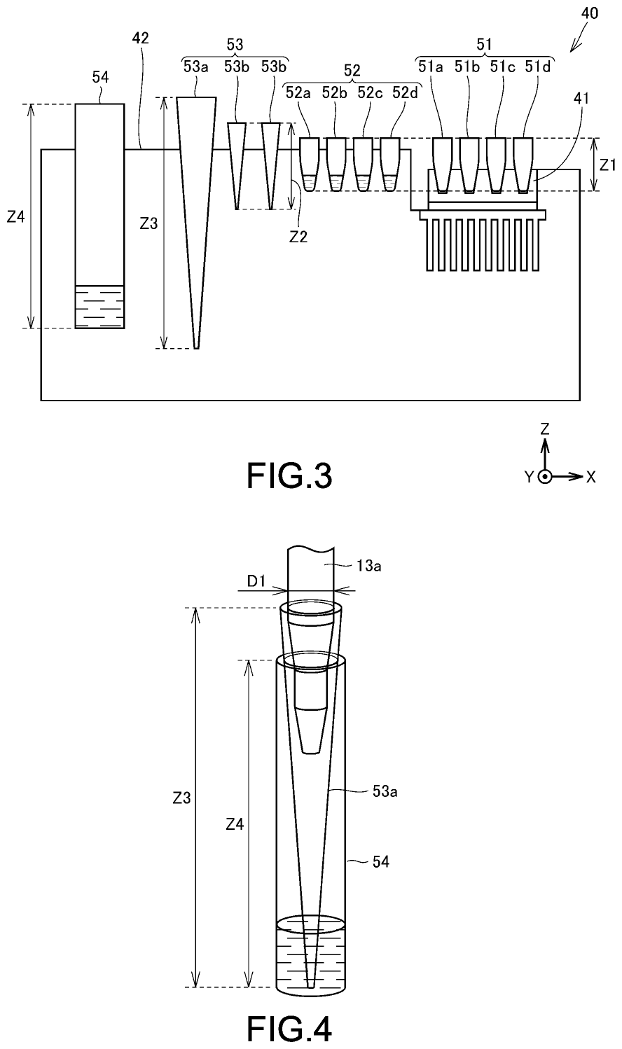

FIG. 3 is a cross section of holding device 40 with container 50 set, as viewed in a direction along the Y-axis. As is also shown in FIG. 2, holding device 40 receives analyte container 54, long pipette tip 53a, two short pipette tips 53b, reagent containers 52a, 52b, 52c and 52d, and PCR containers 51a, 51b, 51c and 51d disposed in this order in the X-axis direction.

PCR container 51 and reagent container 52 are general-purpose containers having the same shape and the same size, and have a height (a dimension in the Z-axis direction) Z1 of the same value (for example about 20 mm). In contrast, analyte container 54 is a container having a size larger than that of PCR container 51 and reagent container 52. Accordingly, analyte container 54 has a height Z4 set to a value larger than height Z1 of PCR container 51 and reagent container 52 (e.g., about 70 mm).

When an analyte is contained in analyte container 54, it is assumed that the analyte is located at a position about 10 to 20 mm above the bottom of analyte container 54 with respect to height Z4 of about 70 mm of analyte container 54. For this reason, if short pipette tip 53b for a small amount for collecting a small amount of several μL is used, short pipette tip 53b would be deeply inserted into analyte container 54, and a possibility that splashes at the time of dispensing adhere not only to short pipette tip 53b but also to nozzle 13a increases, and a possibility of infection or contamination increases.

As a countermeasure therefor, in the present embodiment, short pipette tip 53b is used for PCR container 51 and reagent container 52. Short pipette tip 53b has a height (a dimension in the Z-axis direction) Z2 set to a value larger than height Z1 of PCR container 51 and reagent container 52. In contrast, long pipette tip 53a is used for analyte container 54. Long pipette tip 53a has a height (a dimension in the Z-axis direction) Z3 set to a value larger than height Z4 of analyte container 54.

Further, nozzle 13a of syringe 13 according to the present embodiment is configured to allow both short pipette tip 53b and long pipette tip 53a to be attached thereto.

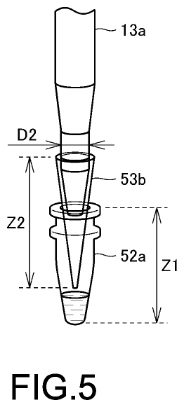

FIG. 4 shows a state in which long pipette tip 53a is attached to nozzle 13a of syringe 13 and thus inserted into analyte container 54. FIG. 5 shows a state in which short pipette tip 53b is attached to nozzle 13a of syringe 13 and thus inserted into reagent container 52a.

As shown in FIG. 4, nozzle 13a is provided with a portion having a diameter D1 adjusted in size to fit a diameter of an opening of long pipette tip 53a. When long pipette tip 53a is attached to nozzle 13a, nozzle 13a is fitted in long pipette tip 53a by inserting nozzle 13a into long pipette tip 53a until the opening of long pipette tip 53a is positioned at the portion of nozzle 13a having diameter D1.

Further, as shown in FIG. 5, a portion of nozzle 13a closer to the tip thereof than the portion having diameter D1 is provided with a portion having a diameter D2 (D2<D1) sized so as to fit a diameter of an opening of short pipette tip 53b. When short pipette tip 53b is attached to nozzle 13a, nozzle 13a is fitted in short pipette tip 53b by inserting nozzle 13a into short pipette tip 53b until the opening of short pipette tip 53b is positioned at the portion of nozzle 13a having diameter D2.

When long pipette tip 53a or short pipette tip 53b fitted to nozzle 13a is detached from nozzle 13a, an upper end of long pipette tip 53a or short pipette tip 53b is hooked on to a lower surface of a recess of pipette tip discarding section 43 and nozzle 13a is moved upward, whereby long pipette tip 53a or short pipette tip 53b is detached from nozzle 13a and discarded.

<Analysis Process>

When an operator sets each container 50 (PCR container 51, reagent container 52, and analyte container 54) and dispensing pipette tip 53 (long pipette tip 53a and short pipette tip 53b) in holding device 40, and inputs a start analysis command to terminal 3 to start an analysis, analysis device 2 starts an analysis process.

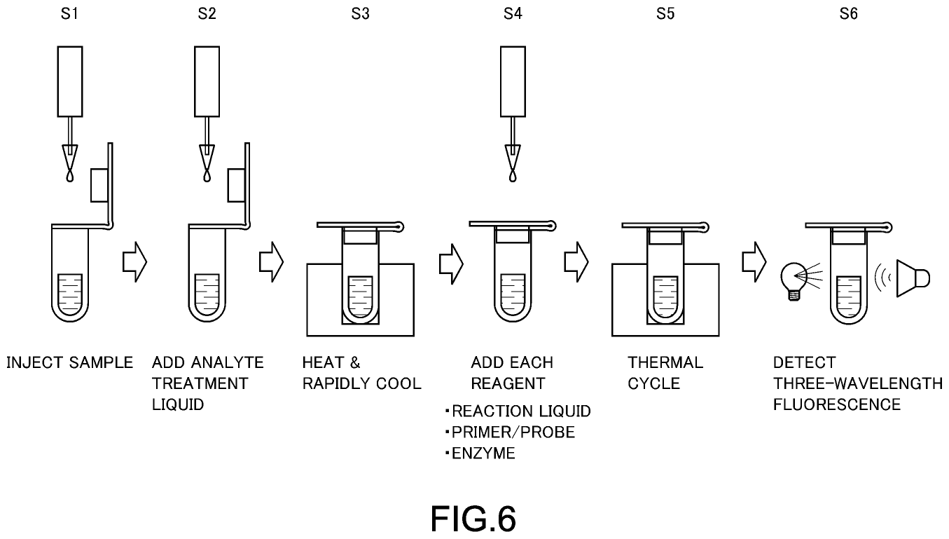

FIG. 6 schematically shows each step of the analysis process performed by analysis device 2. In the analysis process, steps S1 to S6 are executed in this order.

Initially, in step S1, a step of dispensing Slut of an analyte into PCR container 51b (i.e., sample injection) is performed. Specifically, control device 20 initially controls dispensing unit 12 and moving devices 4 and 5 so that long pipette tip 53a is attached to nozzle 13a of syringe 13 and 25 μL of the analyte is collected from analyte container 54 and dispensed into PCR container 51a.

Subsequently, control device 20 controls dispensing unit 12 and moving devices 4 and 5 to discard long pipette tip 53a at pipette tip discarding section 43.

Subsequently, control device 20 controls dispensing unit 12 and moving devices 4 and 5 so that short pipette tip 53b is attached to nozzle 13a of syringe 13 and 5 μL of the analyte is collected from PCR container 51a and dispensed into PCR container 51b.

Note that long pipette tip 53a is used to collect 25 μL of the analyte and temporarily dispense the collected analyte into PCR container 51a and long pipette tip 53a is then replaced with short pipette tip 53b to collect 5 μL of the analyte from PCR container 51a and dispense the collected analyte into PCR container 51b in order to precisely dispense Slut of the analyte into PCR container 51b. In other words, the plunger provided inside nozzle 13a of syringe 13 is thin as it basically corresponds to short pipette tip 53b that dispenses a small amount, and, for the same amount of stroking, using long pipette tip 53a may result in dispensing with reduced precision and hence failing to obtain an accurate result. Accordingly, in the present embodiment, long pipette tip 53a is initially used to once collect an analyte and dispense the collected analyte in an amount of 25 μL, which is larger than 5 μL, into PCR container 51a different from PCR container 51b, and long pipette tip 53a is replaced with short pipette tip 53b, which is used to collect the analyte from PCR container 51a precisely in an amount of Slut and dispense the collected analyte into PCR container 51b. The analyte can thus be dispensed into PCR container 51b precisely in a small amount of 5 μL.

In the next step S2, 5 μL of an analyte treatment liquid is added to PCR container 51b. Specifically, control device 20 initially controls dispensing unit 12 and moving devices 4 and 5 to collect 5 μL of the analyte treatment liquid from reagent container 52a, dispense the Slut of the analyte treatment liquid into PCR container 51b, and reciprocate (or vertically move) syringe 13 to agitate the contents in PCR container 51b.

Subsequently, control device 20 controls dispensing unit 12 and moving devices 4 and 5 to discard short pipette tip 53b in pipette tip discarding section 43.

In the next step S3, PCR container 51b is heated and rapidly cooled. Specifically, control device 20 controls temperature adjustment section 41 so that PCR container 51b is heated to maintain the analyte in PCR container 51b at a temperature of 90° C. for 5 minutes, and thereafter PCR container 51b is rapidly cooled to return the temperature of the analyte in PCR container 51b to 20° C. (or room temperature).

In the next step S4, each reagent is added to PCR container 51b. Specifically, control device 20 controls dispensing unit 12 and moving devices 4 and 5 to initially collect 7.8 μL of a reaction liquid from reagent container 52b and dispense the collected reaction liquid into reagent container 52d containing 2.4 μL of an enzyme in advance.

Subsequently, control device 20 controls dispensing unit 12 and moving devices 4 and 5 to collect 7.8 μL of a primer/probe liquid from reagent container 52c, dispense the collected liquid into reagent container 52d, and reciprocate (or vertically move) syringe 13 to agitate the contents in reagent container 52d. At this point in time, reagent container 52d contains a reagent mixture liquid in an amount of 18 μL.

Subsequently, control device 20 controls dispensing unit 12 and moving devices 4 and 5 to collect 15 μL of the reagent mixture liquid from reagent container 52d, dispenses the 15 μL of the reagent mixture liquid into PCR container 51b, and reciprocate (or vertically move) syringe 13 to agitate the contents in PCR container 51b.

In the next step S5, PCR container 51b undergoes a thermal cycle process. Specifically, control device 20 controls temperature adjustment section 41 to maintain the liquid in PCR container 51b at a temperature of 42° C. for 10 minutes to cause a reverse transcription reaction, and thereafter maintain the liquid in PCR container 51b at a temperature of 95° C. for 1 minute to activate the enzyme.

Subsequently, control device 20 controls temperature adjustment section 41 to maintain the liquid in PCR container 51b at a temperature of 95° C. for 5 seconds and thereafter maintain the liquid in PCR container 51b at a temperature of 60° C. for 30 seconds to perform an amplification process to amplify a gene. This amplification process is performed for 45 cycles.

In the next step S6, three-wavelength fluorescence detection is performed. Specifically, after the amplification process, control device 20 controls temperature adjustment section 41 and optical unit 11 to subject the liquid in PCR container 51b to three-wavelength fluorescence detection while setting the liquid in PCR container 51b at a temperature of 60° C. The three-wavelength fluorescence detection is performed whenever the amplification process is performed. A result of the three-wavelength fluorescence detection (a result of the analysis process by analysis device 2) is displayed on a display of terminal 3.

<Opening and Closing the Container>

In each of steps S1 to S6 of the above-described analysis process, whenever a liquid is collected from each container 50 or dispensed thereinto, an opening operation (an operation of opening the lid) and a closing operation (an operation of closing the lid) are performed for each container 50. As described above, analysis device 2 according to the present embodiment includes opening/closing unit 14 including an opening/closing mechanism for automatically opening/closing the lid of each container 50 by coming into contact with the lid of each container 50. In each of steps S1 to S6, control device 20 controls opening/closing unit 14 so as to automatically open and close the lid of each container 50, as timed, to open and close each container 50, as required.

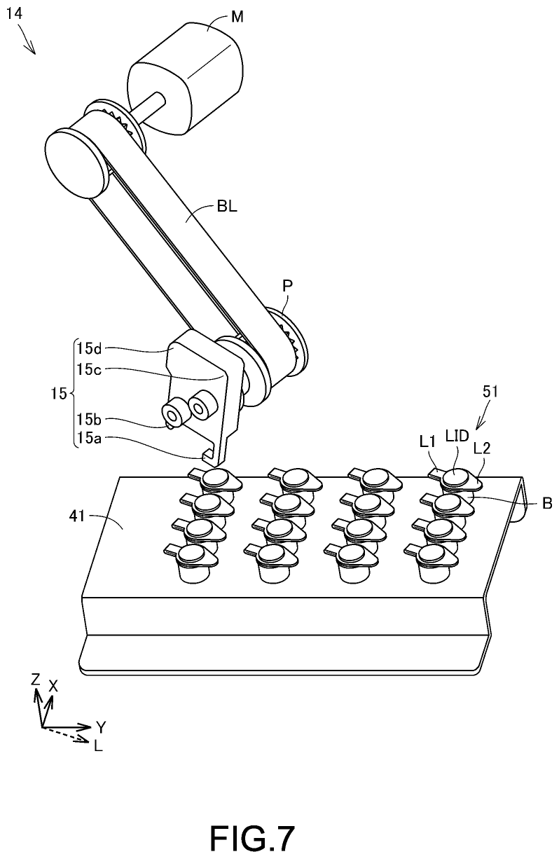

FIG. 7 is a perspective view of the opening/closing mechanism provided to opening/closing unit 14. FIG. 7 shows opening/closing unit 14, and in addition, PCR container 51 held by temperature adjustment section 41 of holding device 40.

PCR container 51 is a resin molded product having a lid LID and a container body B connected to each other by a hinge L1 integrally. Although not shown in FIG. 7, reagent container 52 is a general-purpose container having the same shape and size as PCR container 51, and is, as well as PCR container 51, a resin molded product having lid LID and container body B connected to each other by hinge L1 integrally.

Lid LID is configured to be openable and closable with respect to an opening of container body B. Hinge L1 is formed by bending a portion that interconnects lid LID and container body B in advance in a closed state. Thus, hinge L1 serves as a hinge when lid LID is opened and closed.

Lid LID has a tip provided with a flange L2 for opening and closing lid LID. When an axis extending in a direction from hinge L1 toward flange L2 while PCR container 51 in the closed state is held by holding device 40 in temperature adjustment section 41 is referred to as an “L axis,” each PCR container 51 is held by holding device 40 with the L axis inclined with respect to the X and Y axes.

Opening/closing unit 14 includes a motor M, a belt BL, a pulley P, and a rotating member 15. Rotating member 15 is fixed to a rotating shaft of pulley P. Rotating member 15 has a rotation angle adjusted, as desired, by rotating pulley P via belt BL as motor M is driven. Rotating member 15 has a rotation axis orthogonal to the above-described L axis.

A relative position of rotating member 15 and each PCR container 51 in the X- and Y-axis directions (or a horizontal direction) can be adjusted by control device 20 controlling moving devices 4 and 5.

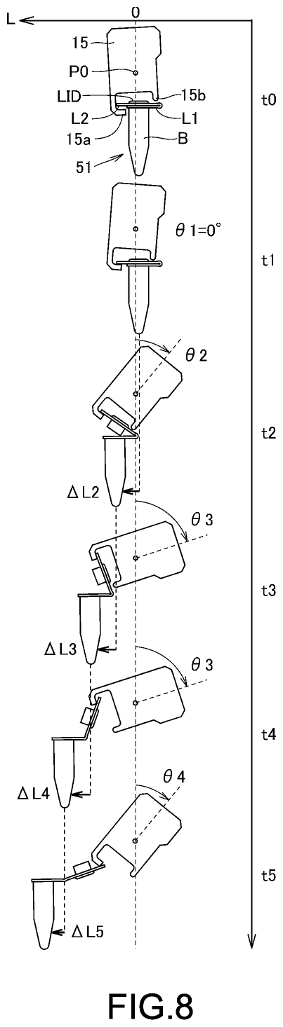

Rotating member 15 includes projections 15a and 15b and end portions 15c and 15d. Projection 15a is formed in an L shape so as to be able to hook on to flange L2 of lid LID. Projection 15b is configured to abut against an upper surface of hinge L1 of lid LID while projection 15a is hooked on to flange L2 of lid LID (or a tip of projection 15a abuts against a lower surface of flange L2 of lid LID) (see FIG. 8 described later).

FIG. 8 schematically shows an operation of rotating member 15 and PCR container 51 when an opening operation for PCR container 51 (an operation of opening lid LID of PCR container 51) is performed. In FIG. 8, a horizontal axis represents a position in the L-axis direction, and a vertical axis represents time. Note that a position in the L-axis direction is indicated with an origin (0) represented by a position of a rotation axis P0 of rotating member 15.

At time t0, initially, control device 20 relatively moves rotating member 15 with respect to PCR container 51 in a direction along rotation axis P0 while adjusting a rotation angle of rotating member 15 so that the tip of projection 15a of rotating member 15 is positioned immediately below flange L2 of lid LID and the tip of projection 15b of rotating member 15 is positioned immediately above hinge L1 of lid LID.

At a subsequent time t1, projections 15a and 15b of rotating member 15 are set on lid LID of PCR container 51. Specifically, control device 20 causes rotating member 15 to rotate clockwise from the state at time t0 to cause the tip of projection 15a of rotating member 15 to abut against the lower surface of flange L2 of lid LID and cause projection 15b of rotating member 15 to abut against the upper surface of hinge L1 of lid LID. In the following description, a rotation angle θ1 of rotating member 15 at time t1 is 0°, and a clockwise rotation angle is a positive rotation angle.

At a subsequent time t2, control device 20 rotates rotating member 15 clockwise (in a direction in which projection 15a lifts flange L2 of lid LID) to set the rotation angle of rotating member 15 to a predetermined angle θ2 (θ2 >θ1). In doing so, as rotating member 15 is further rotated, projection 15a lifts flange L2 of lid LID to displace lid LID in a direction to open it, while projection 15b will serve as a stopper to press a vicinity of hinge L1 of lid LID. This allows lid LID to be opened while suppressing lifting of PCR container 51 per se from temperature adjustment section 41 of holding device 40.

Further, control device 20 controls moving devices 4 and 5 so that, in conjunction with driving and thus rotating rotating member 15, PCR container 51 positionally moves relative to its position at time t1 by a predetermined distance ΔL2 in the L-axis direction. Thus, while projection 15a is displaced in a positive direction along the L-axis as rotating member 15 rotates clockwise, flange L2 of lid LID will also be displaced in the same direction to suppress detachment of projection 15a from flange L2.

At a subsequent time t3, control device 20 further rotates rotating member 15 clockwise to set the rotation angle of rotating member 15 to a predetermined angle θ3 (θ2<θ3<90°). In doing so as well, control device 20 controls moving devices 4 and 5 so that, in conjunction with driving and thus rotating rotating member 15, PCR container 51 positionally moves relative to its position at time t2 by a predetermined distance ΔL3 in the L-axis direction. This suppresses detachment of projection 15a from flange L2.

At a subsequent time t4, control device 20 controls moving devices 4 and 5 so that PCR container 51 positionally moves relative to its position at time t3 by a predetermined distance ΔL4 in the L-axis direction. Thus, lid LID is opened to a greater extent, and an opening degree of lid LID (an angle formed by a plane of an opening of container body B and lid LID) can be set to a value larger than 90°. Further, control device 20 maintains the rotation angle of rotating member 15 at the same predetermined angle θ3 as time t3. This suppresses further upward displacement of projection 15a caused by further clockwise rotation of rotating member and hence suppresses detachment of projection 15a from flange L2.

At a subsequent time t5, control device 20 rotates rotating member 15 counterclockwise (in a direction in which projection 15a pushes down flange L2 of lid LID) to set the rotation angle of rotating member 15 to a predetermined angle θ4 (0°<θ4<θ3). In doing so as well, control device 20 controls moving devices 4 and 5 so that, in conjunction with driving and thus rotating rotating member 15, PCR container 51 positionally moves relative to its position at time t4 by a predetermined distance ΔL5 in the L-axis direction. Thereby, the opening degree of lid LID can be larger than that at time t4 (a value close to 180°). Thus, by increasing an opening degree of lid LID, hinge L1 bent in the closed state can be sufficiently extended, and even when rotating member 15 is detached from lid LID, excessive returning of lid LID in a direction to close it (or lid LID having an opening degree less than 90°) can be suppressed.

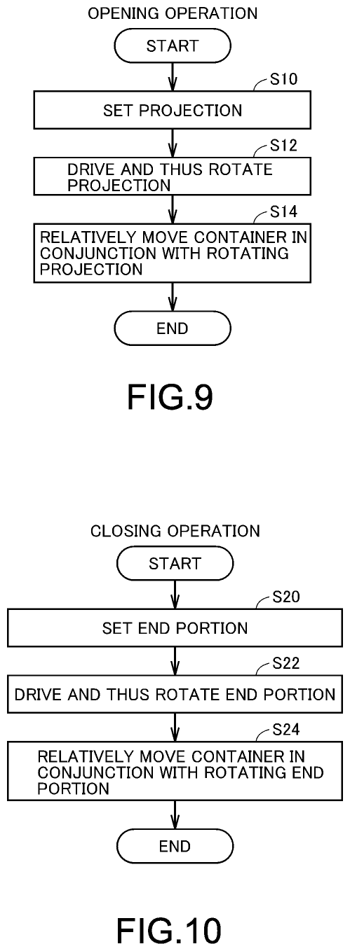

FIG. 9 is a flowchart of an example of a processing procedure when control device 20 performs an opening operation for container 50 (PCR container 51 or reagent container 52). This flowchart is executed for each container 50, as timed, to perform opening each container 50 in each of steps S1 to S6 of the analysis process, as required.

Initially, control device 20 sets projections 15a and 15b of rotating member 15 to lid LID of container 50 (step S10). Specifically, as described above, control device 20 controls opening/closing unit 14 and moving devices 4 and 5 so that the tip of projection 15a of rotating member 15 abuts against the lower surface of flange L2 of lid LID and projection 15b of rotating member 15 abuts against the upper surface of hinge L1 of lid LID.

Subsequently, control device 20 controls opening/closing unit 14 to drive and thus rotate rotating member 15 through a predetermined procedure described with reference to FIG. 8 (step S12).

Subsequently, control device 20, in conjunction with driving and rotating rotating member 15, controls moving devices 4 and 5 to relatively move container 50 in the L-axis direction through the predetermined procedure described with reference to FIG. 8 (step S14).

Thus, analysis device 2 according to the present embodiment includes an opening apparatus capable of opening PCR container 51 or reagent container 52 that is a general-purpose commercially available container without depending on an operator's manual operation. The opening apparatus comprises holding device 40 that holds container body B, opening/closing unit 14, moving devices 4 and 5, and control device 20.

Opening/closing unit 14 includes rotating member 15 having projection 15a (a first projection) and projection 15b (a second projection) that can come into contact with lid LID of the container, and motor M (a drive device) that rotates rotating member 15. Rotating member is rotated, with projections 15a and 15b in contact with lid LID in a closed state, to apply a force to lid LID in a direction to open lid LID. Moving devices 4 and 5 move at least one of holding device 40 and rotating member 15 relative to the other.

Control device 20 controls opening/closing unit 14 and moving devices 4 and 5 in conjunction with each other to open lid LID of the container. Specifically, in conjunction with rotating rotating member 15 of opening/closing unit 14, moving devices 4 and 5 are controlled so that container 50 moves horizontally in the L-axis direction (a direction from hinge L1 of the container toward flange L2 of the container). This allows a general-purpose commercially available container to be appropriately opened without depending on an operator's manual operation.

Further, analysis device 2 according to the present embodiment can also perform a closing operation for container 50 (an operation of closing lid LID of container 50).

FIG. 10 is a flowchart of an example of a processing procedure when control device 20 performs the closing operation for container 50. This flowchart is executed for each container 50, as timed, to perform closing each container 50 in each of steps S1 to S6 of the analysis process, as required.

Initially, control device 20 sets end portion 15d of rotating member 15 on lid LID of container 50 (step S20). Specifically, control device 20 controls opening/closing unit 14 and moving devices 4 and 5 so that end portion 15d of rotating member 15 abuts around the center of the upper surface of lid LID in the open state.

Subsequently, control device 20 controls opening/closing unit 14 to drive and thus rotate rotating member 15 in a closing direction (a direction to close lid LID) through a predetermined procedure (step S22).

Subsequently, control device 20 controls moving devices 4 and 5 to relatively move container 50 in the L-axis direction in conjunction with driving and thus rotating rotating member 15 (step S24). For example, when end portion 15d of rotating member 15 is displaced in the positive direction along the L-axis by driving and thus rotating rotating member 15, container 50 is relatively moved in the L-axis direction in accordance with the displacement of end portion 15d to prevent end portion 15d of rotating member 15 from being displaced from around the center of the upper surface of lid LID. Thus, the closing operation for container 50 can be performed appropriately.

When lid LID of container 50 is closed after the state shown in FIG. 8 at time t5, control device 20 may perform the following closing operation:

Initially, control device 20 releases lid LID from rotating member 15 by moving PCR container 51 further in the positive direction along the L-axis (a leftward direction in FIG. 8) from the state shown in FIG. 8 at time t5. Lid LID returns in the closing direction by the elastic force of hinge L1, and stands erect to be generally perpendicular to the plane of the opening of container body B (or lid LID has an opening degree of substantially 90°).

After that, control device 20 rotates rotating member 15 so that end portion 15d of rotating member 15 protrudes downward (below projections 15a and 15b and end portion 15c). After that, control device 20 relatively moves PCR container 51, with lid LID standing erect substantially vertically, in a rightward direction in FIG. 8 toward end portion 15d of rotating member 15. As a result, lid LID collides against end portion 15d protruding downward, and is thus pushed downward and closed. Such a closing operation may be performed.

As described above, analysis device 2 according to the present embodiment can automatically open and close a general-purpose commercially available container without depending on an operator's manual operation. Further, analysis device 2 according to the present embodiment fully automatically performs a gene analysis through PCR, including collecting and dispensing an analyte and a reagent. The operator does not need to perform the opening/closing operation for the container and the operation of collecting and dispensing the analyte and the reagent, and poor analysis due to contamination can be reduced and quick analysis can be performed. Furthermore, the operator does not need to be skilled, and a PCR test can be simply and quickly performed even in small- and medium-sized hospitals, clinics and other similar medical institutions having few opportunities to perform the PCR test.

<Configuration of Irradiance Unit 16>

In the heating and rapid cooling step in step S3 of the analysis process described above, the lid of PCR container 51b is closed. In the reagent adding step in step S4 after the heating and rapid cooling step, PCR container 51b is opened by rotating rotating member 15 while projection 15a of rotating member 15 is hooked on to flange L2 of lid LID. In doing so, when the analyte in PCR container 51b splashes and adheres to projection 15a of rotating member 15, the analyte may contaminate a next analyte.

As a countermeasure therefor, analysis device 2 according to the present embodiment includes an irradiation unit 16 that prevents contamination by irradiating projection 15a of opening/closing unit 14 and a vicinity thereof with UV light (ultraviolet ray).

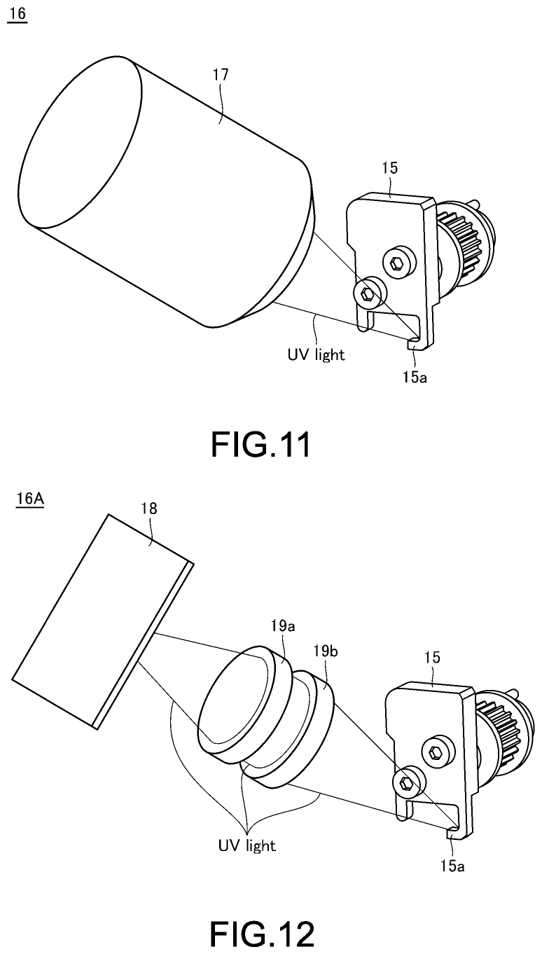

FIG. 11 shows an example of a configuration of irradiation unit 16. Irradiation unit 16 shown in FIG. 11 includes a UV light source unit 17 that generates UV light (ultraviolet ray) and directly irradiates projection 15a of opening/closing unit 14 and a vicinity thereof therewith.

FIG. 12 shows an exemplary variation of the configuration of irradiation unit 16. Irradiation unit 16A shown in FIG. 12 includes a UV light source substrate 18 having a light emitting diode that generates UV light, and lenses 19a and 19b that collect UV light generated by UV light source substrate 18 to irradiate projection 15a of opening/closing unit 14 and a vicinity thereof therewith.

Control device 20 controls irradiation unit 16 and moving devices 4 and 5 so that in each of steps S1 to S6 of the analysis process described above, after at least one of projection 15a and projection 15b of rotating member 15 is brought into contact with lid LID of container 50 with an analyte contained therein, at least one of the projections brought into contact with lid LID is partially or entirely irradiated with UV light. In this way, even when the analyte splashes and adheres to projections 15a and 15b of rotating member 15, the UV light can sterilize in advance viruses present in the analyte and having adhered to projections 15a and 15b or inactivate genes in advance, and thus prevent a next analyte from being contaminated therewith.

<Variation of opening/closing unit 14>

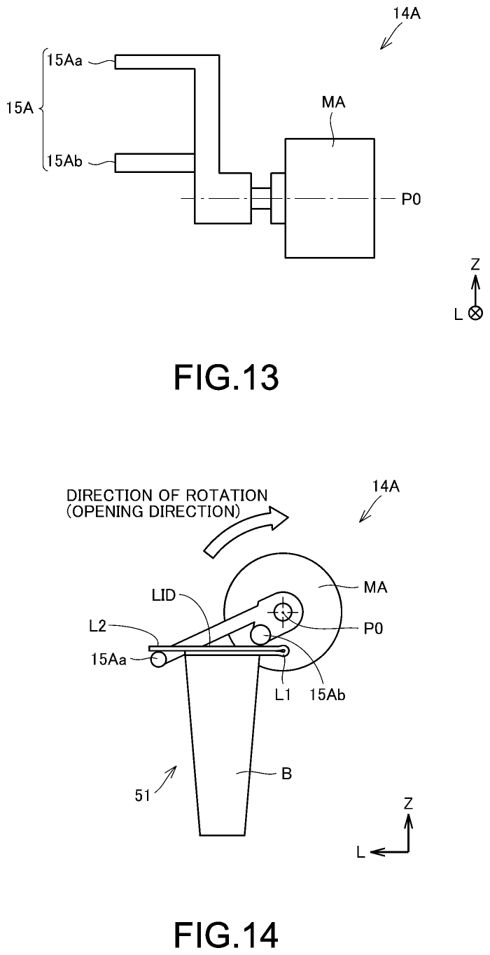

FIG. 13 shows an exemplary variation of the configuration of opening/closing unit 14. Opening/closing unit 14A shown in FIG. 13 includes a motor MA and a rotating member 15A. Rotating member 15A is fixed along rotation axis P0 of motor MA. Rotation angle of rotating member 15A is adjusted, as desired, by driving motor MA.

Rotating member 15A has two projections 15Aa and 15Ab extending in a direction orthogonal to the L axis. Projection 15Aa is disposed at a position farther from rotation axis P0 than projection 15Ab is. Projections 15Aa and 15Ab of rotating member 15A correspond to projections 15a and 15b, respectively, of rotating member 15.

FIG. 14 shows a state in which projections 15Aa and 15Ab of rotating member 15A are set on lid LID of PCR container 51 in the opening operation for PCR container 51. As shown in FIG. 14, when PCR container 51 is opened, a tip of projection 15Aa of rotating member 15A is set to abut against a lower surface of flange L2 of lid LID, and projection 15Ab of rotating member 15A is set to abut against an upper surface of hinge L1 of lid LID. In this state, when rotating member 15A is rotated in an opening direction (a direction in which projection 15Aa lifts flange L2 of lid LID), projection 15Aa lifts flange L2 of lid LID to displace lid LID in the opening direction, whereas projection 15Ab serves as a stopper to press a vicinity of hinge L1 of lid LID. Thus, as well as rotating member 15 described above, rotating member 15A can open lid LID without lifting PCR container 51 itself from holding device 40.

Further, PCR container 51 is positionally, relatively moved in the L-axis direction in conjunction with driving and thus rotating rotating member 15A. Thus, even if projection is displaced as rotating member 15A rotates, flange L2 of lid LID can be displaced in the positive direction along the L-axis in accordance with the displacement of projection 15Aa to suppress detachment of projection 15Aa from flange L2.

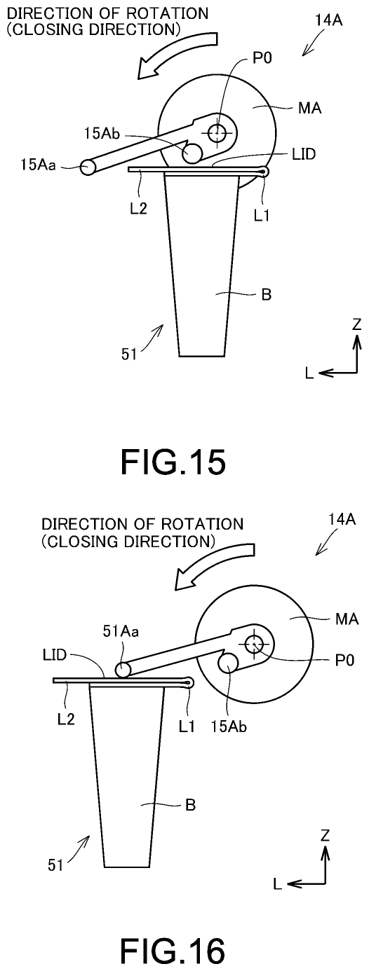

FIG. 15 shows a state in which lid LID of PCR container 51 is closed by using projection of rotating member 15A. In the closing operation for PCR container 51, in a state in which projection 15Ab of rotating member 15A is set to abut against a vicinity of the center of the upper surface of lid LID, lid LID is closed by rotating rotating member 15A in the closing direction.

Further, relatively moving PCR container 51 in the L-axis direction in conjunction with driving and thus rotating rotating member 15A can prevent projection 15Ab of rotating member from being displaced from the vicinity of the center of the upper surface of lid LID. The closing operation for PCR container 51 can thus be appropriately performed.

Container 50 can be closed by using projection 15Aa of rotating member 15A, rather than projection 15Ab thereof.

FIG. 16 shows a state in which lid LID of PCR container 51 is closed by using projection of rotating member 15A. In the closing operation for PCR container 51, in a state in which projection 15Ab of rotating member 15A is set to abut against a vicinity of the center of the upper surface of lid LID, lid LID is closed by rotating rotating member 15A in the closing direction.

Further, relatively moving PCR container 51 in the L-axis direction in conjunction with driving and thus rotating rotating member 15A can prevent projection 15Ab of rotating member from being displaced from the vicinity of the center of the upper surface of lid LID. The closing operation for PCR container 51 can thus be appropriately performed.

<Reagent Kit>

Hereinafter, a reagent kit provided with four reagent containers 52a, 52b, 52c and 52d as one set will be described. Some PCR tube used for reagent container 52 is commercially available in a state with eight containers connected to one another.

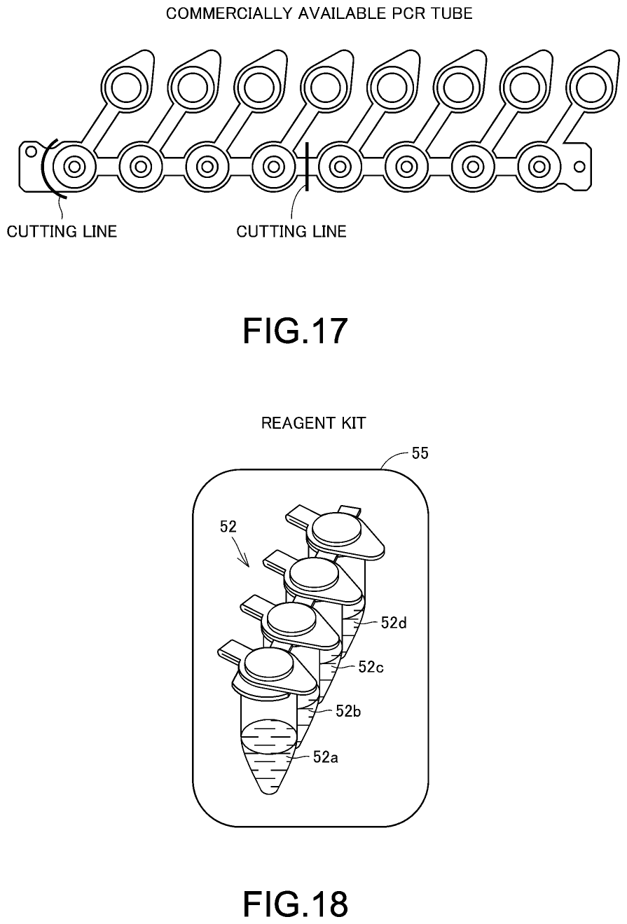

FIG. 17 shows an external appearance of a commercially available 8-gang PCR tube. As shown in FIG. 17, there is a commercially available PCR tube having eight containers connected one-dimensionally.

In the present embodiment, such a commercially available 8-gang tube is cut into two sets each of a 4-gang tube. Further, a grip portion of one end portion (the left end portion in FIG. 17) of the commercially available tube is also cut as it is unnecessary.

Four reagent containers 52a, 52b, 52c and 52d are produced and provided as a reagent kit by sealing an analyte treatment liquid, a reaction liquid, a primer/probe liquid, and an enzyme liquid in the four containers, respectively, of the 4-gang tube in an amount necessary for analysis of at least one analyte.

FIG. 18 schematically shows an appearance of a reagent kit. In this reagent kit, as shown in FIG. 18, four reagent containers 52a, 52b, 52c and 52d disposed in a one-dimensional manner and each connected to an adjacent one thereof are packaged in a packaging material 55 and thus provided. Each lid of the four reagent containers 52a, 52b, 52c and 52d is closed. Packaging material 55 may be a box made of paper or a bag made of resin.

Such a reagent kit allows an operator to efficiently start an analysis process. That is, rather than setting mutually separated reagent containers 52a, 52b, 52c and 52d in holding device 40, simply extracting one-dimensionally connected reagent containers 52a, 52b, 52c and 52d from packaging material 55 and setting the reagent containers in holding device 40 can complete an operation of setting reagent containers 52.

Further, as another consumable product, a 4-gang PCR container 51 and dispensing pipette tip 53 (long pipette tip 53a and short pipette tip 53b) may be sold in combination.

<Controlling Opening Angle of Lid LID>

When the opening operation shown in FIG. 8 is completed and thereafter projection 15a of rotating member 15 detaches from lid LID, rotating member 15 no longer constrains lid LID, and accordingly, lid LID returns in the closing direction and thereafter will stop. When lid LID after it returns in the closing direction after the opening operation ends has a degree of opening (hereinafter also simply referred to as an “opening angle after the end of the opening operation”) that is excessively small, and dispensing pipette tip 53 is subsequently used to perform dispensing into container 50, dispensing pipette tip 53 may interfere with lid LID of container 50. In contrast, when the opening angle after the end of the opening operation is an excessively large angle, then, thereafter, other members (such as a contact lever sensor for confirming that the container is opened/closed, an opener, etc.) may interfere with lid LID of container 50. Therefore, it is desirable that the opening angle after the end of the opening operation is an optimal opening degree (approximately 90°) at which interference between lid LID and other components (e.g., dispensing pipette tip 53, the contact lever sensor, and the opener) can be avoided.

As shown in FIG. 6, in the analysis process by analysis device 2, PCR container 51b is heated in step S3. As the treatment of step 3 is performed with lid LID closed, lid LID is snugger in the closed state after the heating in step 3 than therebefore. Due to this effect, an amount by which lid LID returns in the closing direction after the opening operation ends as rotating member 15 no longer constrains the lid is larger after the heating in step 3 than therebefore. In view of this point, it is possible to uniformly set a large opening angle for a point in time when the opening operation ends so that the opening angle after the end of the opening operation after the heating in step 3 is an optimal opening degree (of approximately 90°). In this case, however, lid LID before the heating in step 3 returns in the closing direction in a small amount, and accordingly, the opening angle after the end of the opening operation would be larger than approximately 90°, and interference with other components may no longer be avoidable.

Accordingly, control device 20 sets an opening angle at a point in time when the opening operation ends before the heating in step 3 and an opening angle at a point in time when the opening operation ends after the heating in step 3 to mutually different values. Specifically, when performing the opening operation before the heating in step 3, control device 20 controls opening/closing unit 14 and moving devices 4 and 5 so that the opening angle at a point in time when the opening operation ends is a first angle α. In contrast, when performing the opening operation after the heating in step 3, control device 20 controls opening/closing unit 14 and moving devices 4 and 5 so that the opening angle at a point in time when the opening operation ends is a second angle β larger than first angle α. Thus, both before and after the heating in Step 3, the opening angle after the end of the opening operation can be set to an optimal opening degree capable of avoiding interference between lid LID and other components such as dispensing pipette tip 53.

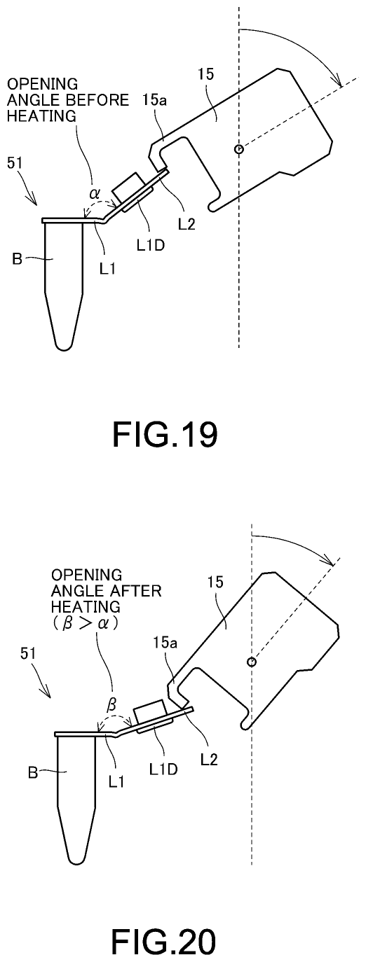

FIG. 19 schematically shows a state of rotating member 15 and PCR container 51 at a point in time when the opening operation ends before the heating in Step 3. Before the heating in step 3, in view of the fact that lid LID after the opening operation ends returns in the closing direction in a relatively small amount, as shown in FIG. 19, the opening angle at a point in time when the opening operation ends is suppressed to a relatively small, first angle α (for example of about 120°). When this is compared with setting the opening angle after the end of the opening operation to second angle β that is larger than first angle α (for example, about 160°), the former can suppress an excessively large opening angle after the end of the opening operation, and allows an optimal opening degree (of approximately 90°). This can help to suppress interference of lid LID with other components in a subsequent step.

FIG. 20 schematically shows a state of rotating member 15 and PCR container 51 at a point in time when the opening operation ends after the heating in Step 3. After the heating in step 3, in view of the fact that lid LID after the opening operation ends returns in the closing direction in a relatively large amount, the opening angle at a point in time when the opening operation ends is set to second angle β larger than first angle α (for example of about 160°). When this is compared with setting the opening angle after the end of the opening operation to first angle α (for example, about 120°), the former can suppress an excessively small opening angle after the end of the opening operation, and allows an optimal opening degree (of approximately 90°). This can help to suppress interference of lid LID with other components in a subsequent step.

<Dispensing Operation by Dispensing Pipette Tip 53>

By controlling an opening angle, as described above, the opening angle after the end of the opening operation can be set to an optimal opening degree (approximately 90°) at which interference between lid LID and other components can be avoided. However, depending on the individual difference of container 51, it is also expected that the opening angle after the end of the opening operation would be smaller than the optimal opening degree (approximately 90°).

In view of this point, when dispensing into container 50 is performed, control device 20 controls moving devices 4 and 5 so that dispensing pipette tip 53 moves horizontally from the side of flange L2 (or the side of the tip) of lid LID of container 50 toward the side of hinge L2 (or the side of the proximal end) thereof.

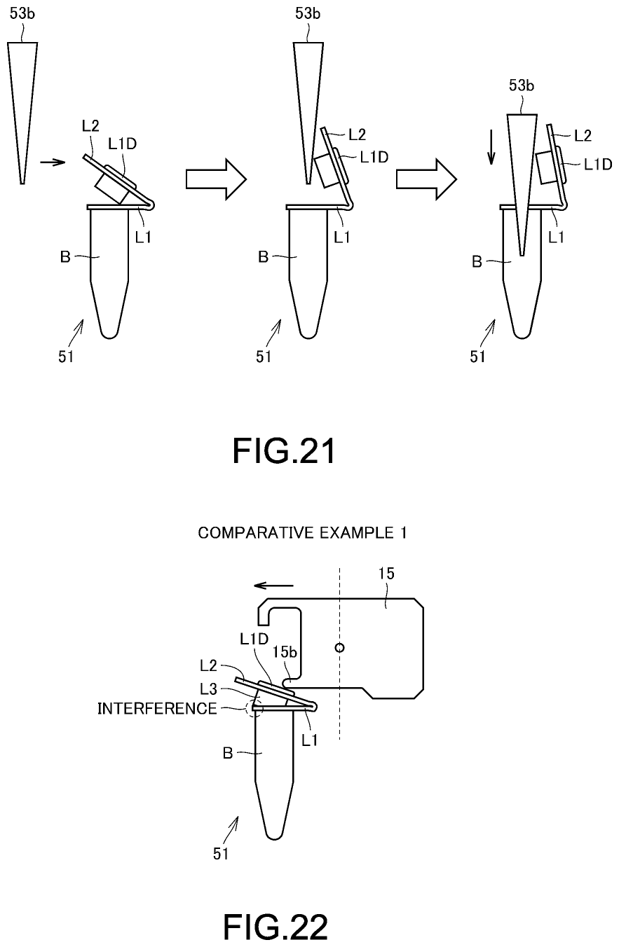

FIG. 21 schematically shows movement of short pipette tip 53b with respect to PCR container 51 when dispensing into PCR container 51 is performed using short pipette tip 53b. As shown in FIG. 21, when dispensing into PCR container 51, control device 20 controls moving devices 4 and 5 so that dispensing pipette tip 53 moves horizontally from the side of flange L2 (or the side of the tip) of lid LID of PCR container 51 toward the side of hinge L2 (or the side of the proximal end) thereof in a state where a position of the tip portion of dispensing pipette tip 53 in the vertical direction is aligned with a position of lid LID of PCR container 51 in the vertical direction. As a result, even when the opening angle after the end of the opening operation is a small angle and lid LID is closing to some extent, short pipette tip 53b horizontally moved can be brought into contact with lid LID to set lid LID in an opened state. Thus, after that, short pipette tip 53b can be inserted into PCR container 51.

<Countermeasure Against Insufficient Closing of Container>

A protrusion L3 is provided on the back surface of lid LID of container 50 (see FIG. 22 etc. described later). Container 50 is closed by inserting and fitting protrusion L3 into container body B. When the closing operation for container 50 is inappropriately performed, it is expected that protrusion L3 of lid LID interferes with the edge of the opening of container body B and container 50 cannot be closed appropriately.

FIG. 22 schematically shows a closing operation according to comparative example 1. In the closing operation according to comparative example 1, rotating member 15 is relatively moved in a horizontal direction so that projection 15b of rotating member 15 approaches flange L2 (or the tip) of lid LID while the projection is in contact with lid LID. In such a closing operation, rotating member 15 displaced toward flange L2 in contact with lid LID pushes lid LID toward flange L2, and would displace lid LID positionally toward flange L2 with respect to container body B. As a result, the tip of protrusion L3 of lid LID may interfere with the edge of the opening of container body B and protrusion L3 may not be able to be fitted to container body B.

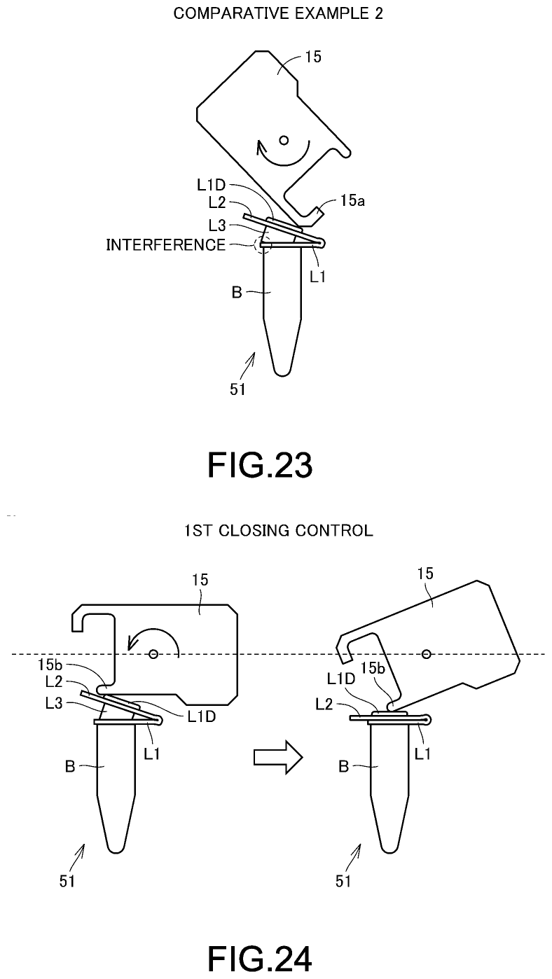

FIG. 23 schematically shows a closing operation according to comparative example 2. In the closing operation according to comparative example 2, rotating member 15 is rotated so that projection 15a of rotating member 15 approaches the tip of lid LID in contact with lid LID. In such a closing operation as well, as well as comparative example 1, rotating member 15 displaced toward flange L2 in contact with lid LID pushes lid LID toward flange L2, and would displace lid LID positionally toward flange L2 with respect to container body B. As a result, the tip of protrusion L3 of lid LID may interfere with the edge of the opening of container body B and protrusion L3 may not be able to be fitted to container body B.

In order to prevent such insufficient closing, when container 50 in the open state is brought to the closed state, control device 20 according to the present embodiment continuously performs mutually different types of first opening control and second closing control in this order. Hereinafter, the first opening control and the second closing control will be described in detail.

(First Closing Control)

FIG. 24 schematically shows an operation by the first closing control. In the first closing control, rotating member 15 with projection 15b thereof in contact with lid LID at a location closer to flange L2 than a location in a vicinity of the center of the upper surface of lid LID, is rotated to move toward and thus approach hinge L1 of lid LID (that is, a side opposite to flange L2) while projection 15b is in contact with lid LID. This pushes lid LID toward hinge L1, rather than toward flange L2, and thus suppresses positional displacement of lid LID toward flange L2. As a result, interference between protrusion L3 and container body B does not occur, and protrusion L3 can be appropriately fitted to container body B.

In the first closing control, the portion of rotating member 15 to be brought into contact with lid LID is not necessarily limited to projection 15b. For example, projection 15a or end portion 15d of rotating member 15 may be brought into contact with lid LID.

(Second Closing Control)

In the first closing control described above, a force is caused to act to push lid LID toward hinge L1 and accordingly, a force that presses lid LID from above is slightly weakened, and it is thus expected that lid LID would be slightly opened. Accordingly, control device 20 according to the present embodiment successively performs the second closing control after executing the first opening control.

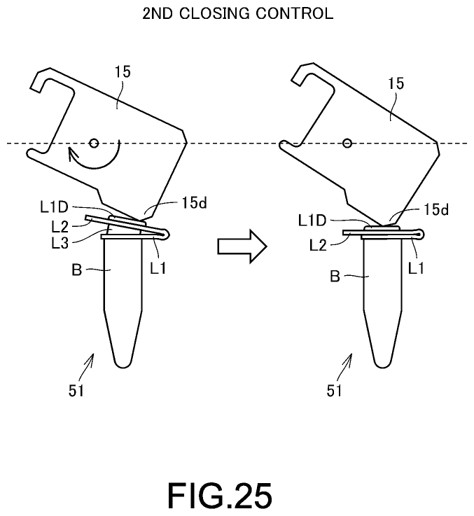

FIG. 25 schematically shows an operation by the second closing control. In the second closing control, rotating member 15 is rotated so that end portion 15d of rotating member 15 moves toward flange L2 of lid LID in contact with lid LID. The second closing control can firmly press lid LID from above, and more reliably close lid LID even when lid LID is slightly opened after the first closing control is executed.

After the first closing control is executed, the tip of protrusion L3 of lid LID is already inserted into container body B. Therefore, in the second closing control, even if a force acts to push lid LID toward flange L2, interference between protrusion L3 and container body B does not occur, and protrusion L3 can be appropriately fitted to container body B.

In the second closing control, the portion of rotating member 15 to be brought into contact with lid LID is not necessarily limited to end portion 15d. For example, projection 15a or projection 15b of rotating member 15 may be brought into contact with lid LID.

Further, the second closing control is not limited to rotating rotating member 15. For example, by controlling a device that moves rotating member 15 relative to container 50 in an upward/downward direction, rotating member 15 may be displaced downward while rotating member 15 is in contact with lid LID.

[Aspects]

It is understood by those skilled in the art that the above-described embodiments and variations thereof are specific examples of the following aspects.

(Clause 1) An opening apparatus according to an aspect is an opening apparatus for a container including a container body, a lid, and a hinge that interconnects the container body and the lid. The opening apparatus comprises: a holding device that holds the container body; an opening device including a rotating member and a drive device that rotates the rotating member, the rotating member having a first projection and a second projection capable of coming into contact with the lid, the rotating member being configured to apply a force to the lid in a direction to open the lid by rotating while one of the first and second projections is in contact with the lid in a closed state; a moving device that moves at least one of the holding device and the rotating member relatively to the other; and a control device that controls the opening device and the moving device in conjunction with each other to open the lid.

According to the opening apparatus of clause 1, when the rotating member rotates while at least one of the first and second projections is in contact with the lid of the container, a force is applied to the lid in a direction to open the lid. Further, in conjunction with rotation of the rotating member, the holding device holding the container body is relatively moved with respect to the rotating member. Accordingly, the container can be positionally displaced in accordance with positional displacement of the first and second projections with the rotation of the rotating member. Therefore, even if the first and second projections are positionally displaced as the rotating member rotates, the first and second projections no longer coming into contact with the lid can be suppressed. As a result, a general-purpose commercially available container can be appropriately opened without depending on an operator's manual operation.

(Clause 2) For the opening apparatus for a container according to clause 1, a flange is provided at a tip of the lid. The control device controls the moving device so that a set state is set in which while the container body is held by the holding device and the lid is positioned above the container body, the first projection of the rotating member is in contact with a lower surface of the flange of the lid and the second projection of the rotating member is in contact with an upper surface of the hinge of the lid, and the control device controls the opening device in the set state so that the rotating member rotates in a direction to displace the flange upward.

According to the opening apparatus of clause 2, as the rotating member rotates, the first projection displaces the flange of the lid upwards to displace the lid in a direction to open it, while the second projection will serve as a stopper to press a vicinity of the hinge of the lid. This can open the lid while suppressing lifting of the container per se from the holding device.

(Clause 3) In the opening apparatus according to clause 2, in conjunction with rotating the rotating member, the control device controls the moving device so that the container moves horizontally in a direction from the hinge toward the flange.

According to the opening apparatus of the clause 3, even when the first projection is displaced in a direction from the hinge toward the flange as the rotating member rotates, the flange of the lid can also be displaced in the direction from the hinge toward the flange. This can suppress detachment of the first projection from the flange of the lid that accompanies rotation of the rotating member.

(Clause 4) In the opening apparatus according to any one of clauses 1 to 3, the rotating member has an end portion different from the first and second projections. The end portion applies a force to the lid in a direction to close the lid when the rotating member rotates in the direction to close the lid while the rotating member is in contact with the lid in an open state.

According to the opening apparatus of the clause 4, the lid can be closed by rotating the rotating member in a direction to close the lid while the rotating member has the end portion in contact with the lid.

(Clause 5) In the opening apparatus according to any one clauses 1 to 3, at least one of the first and second projections of the rotating member applies a force to the lid in a direction to close the lid when the rotating member rotates in the direction to close the lid while the rotating member is in contact with the lid in an open state.

According to the opening apparatus of clause 5 the lid can be closed by rotating the rotating member in a direction to close the lid while at least one of the first and second projections of the rotating member is in contact with the lid.

(Clause 6) In the opening apparatus according to any one of clauses 1 to 5, the holding device has an arrangement surface in which a plurality of containers are two-dimensionally disposed. The moving device is configured to two-dimensionally move at least one of the holding device and the rotating member relative to the other along the arrangement surface.

According to the opening apparatus of clause 6, the plurality of containers disposed two-dimensionally in the arrangement surface of the holding device can be appropriately opened.

(Clause 7) The opening apparatus according to any one of clauses 1 to 6 further comprises an irradiation device that irradiates at least one of the first and second projections of the rotating member partially or entirely with ultraviolet ray.

According to the opening apparatus of clause 7, viruses adhering to the first or second projection can be sterilized with an ultraviolet ray emitted by the irradiation device, or genes adhering to the first or second projection can be inactivated with an ultraviolet ray emitted by the irradiation device.

(Clause 8) In the opening apparatus according to clause 7, the control device controls the irradiation device so that, after at least one of the first and second projections is brought into contact with the lid of the container with an analyte contained therein, the at least one of the projections brought into contact with the lid is partially or entirely irradiated with ultraviolet ray.

According to the opening apparatus of Clause 8, even when the first or second projection comes into contact with the lid of the container with an analyte contained therein and accordingly, a virus or the analyte adheres to the projection, the virus or the analyte is sterilized or inactivated by an ultraviolet ray emitted by the irradiation device. This can prevent the virus or gene adhering to the first or second projection from contaminating a next analyte.

(Clause 9) In the opening apparatus according to any one of clauses 1 to 8, the holding device has a function to heat the container. The control device controls the opening device and the moving device so that the lid is opened at a first angle when the lid is brought to an open state before the holding device heats the container, and the control device controls the opening device and the moving device so that the lid is opened at a second angle when the lid is brought to an open state after the holding device heats the container, the second angle being larger than the first angle.

According to the opening apparatus of Clause 9, both before and after the container is heated, the lid after the end of the opening operation can be opened at an angle set optimally to avoid interference with another component.

(Clause 10) According to an aspect an analysis device comprises: an opening apparatus according to any one of clauses 1 to 9; and a dispensing device that dispenses a reagent into a container opened by the opening apparatus.

According to the analysis device of Clause 10, a container can be opened and a reagent can be dispensed into the opened container without depending on an operator's manual operation.

(Clause 11) The analysis device according to Clause 10 further comprises a pipette tip used to dispense liquid into the container. The dispensing device includes a nozzle that allows the pipette tip to be detachable therefrom. The control device controls the moving device so that when liquid is dispensed into the container via the pipette tip attached to the nozzle the pipette tip moves horizontally from a side of a tip of the lid toward the hinge.

According to the analysis device of clause 11, even when the lid of the container is closing, the lid can be opened by the horizontal movement of the pipette tip. Therefore, the pipette tip can be inserted into the container thereafter.

(Clause 12) The analysis device according to clause 10 or 11 analyzes a gene using a polymerase chain reaction.

According to the analysis device of clause 12, in a gene analysis using the polymerase chain reaction, the container can be opened and the reagent can be dispensed into the container without depending on the operator's manual operation.

(Clause 13) According to an aspect, a closing apparatus for a container includes a container body, a lid, and a hinge that interconnects the container body and the lid. The closing apparatus comprises: a holding device that holds the container body; a closing device including a rotating member and a drive device that rotates the rotating member, the rotating member being configured to apply a force to the lid in a direction to close the lid by rotating in contact with the lid in an open state; and a control device that controls the closing device. When the lid in the open state is closed, the control device performs a first closing control to apply a force to the lid in a direction to close the lid by rotating the rotating member so that a portion of the rotating member approaches the hinge while the portion of the rotating member is in contact with the lid.

According to the closing apparatus of Clause 13, the first closing control pushes the lid toward the hinge, rather than toward a tip of the lid, and thus suppresses positional displacement of the lid with respect to the container body toward the tip of the lid. As a result, there is no interference caused between the lid and the container body, and the lid can be appropriately closed.

(Clause 14) The closing apparatus for a container according to clause 13 further comprises a moving device that moves at least one of the holding device and the rotating member relatively to the other. After the first closing control is executed, the control device executes a second closing control to control at least one of the closing device and the moving device so that the rotating member applies a force to the lid to close the lid while the rotating member is partially in contact with the lid.

According to the closing apparatus according to clause 14, even when the lid is slightly opened after the first closing control is executed, the second closing control can more reliably close the lid.

(Clause 15) A reagent kit according to one aspect comprises a plurality of containers and a packaging material that packages the plurality of containers. Each of the plurality of containers includes a container body and a lid connected to the container body via a hinge and capable of opening and closing with respect to the container body. The plurality of containers are provided such that they are one-dimensionally disposed, with adjacent containers connected to each other, the lid closed, and a reagent introduced in each container body.

According to the reagent kit of clause 15, a reagent container setting operation can be completed simply by setting a plurality of one-dimensionally connected containers in a holding device, rather than setting a plurality of mutually separated containers each in the holding device. As a result, reagent containers can be set efficiently in an analysis device.

It should be understood that the presently disclosed embodiments are illustrative and non-restrictive in any respect. The scope of the present invention is defined by the terms of the claims, rather than the above description of the embodiments, and is intended to include any modifications within the meaning and scope equivalent to the terms of the claims.

### REFERENCE SIGNS LIST

1 analysis system, 2 analysis device, 3 terminal 4, 5 moving device, 10 inspection device, 11 optical unit, 12 dispensing unit, 13 syringe, 13a nozzle, 14, 14A, opening and closing unit, 15A, rotating member, 15a, 15b, 15Aa, 15Ab, projection, 15c, 15d, end portion, 16, 16A, irradiation unit, 17 UV light source unit, 18 UV light source substrate, 19a, 19b lens, 20 control device, 30 temperature adjustment device, 40 holding device, 41 temperature adjustment section, 42 holding section, 43 pipette tip discarding section, 50 container, 51, 51a, 51b, 51c, 51d PCR container, 52, 52a, 52b, 52c, 52d reagent container, 53 pipette tip, 53a long pipette tip, 53b short pipette tip, 54 analyte container, 55 packaging material, B container body, BL belt, L1 hinge, L2 flange, L3 protrusion, LID lid, M, MA motor, P pulley, P0 rotation axis.

1. An opening apparatus for a container including a container body, a lid, and a hinge that interconnects the container body and the lid, comprising:
a holding device that holds the container body;
an opening device including a rotating member and a drive device that rotates the rotating member, the rotating member having a first projection and a second projection capable of coming into contact with the lid, the rotating member being configured to apply a force to the lid in a direction to open the lid by rotating while one of the first and second projections is in contact with the lid in a closed state;
a moving device that moves at least one of the holding device and the rotating member relatively to the other; and
a control device that controls the opening device and the moving device in conjunction with each other to open the lid.

2. The opening apparatus for a container according to claim 1, wherein
a flange is provided at a tip of the lid,
the control device controls the moving device so that a set state is set in which while the container body is held by the holding device and the lid is positioned above the container body, the first projection of the rotating member is in contact with a lower surface of the flange of the lid and the second projection of the rotating member is in contact with an upper surface of the hinge of the lid, and
the control device controls the opening device in the set state so that the rotating member rotates in a direction to displace the flange upward.

3. The opening apparatus for a container according to claim 2, wherein, in conjunction with rotating the rotating member, the control device controls the moving device so that the container moves horizontally in a direction from the hinge toward the flange.

4. The opening apparatus for a container according to claim 1, wherein
the rotating member has an end portion different from the first and second projections, and
the end portion applies a force to the lid in a direction to close the lid when the rotating member rotates in the direction to close the lid while the rotating member is in contact with the lid in an open state.

5. The opening apparatus for a container according to claim 1, wherein at least one of the first and second projections of the rotating member applies a force to the lid in a direction to close the lid when the rotating member rotates in the direction to close the lid while the rotating member is in contact with the lid in an open state.

6. The opening apparatus for a container according to claim 1, wherein
the holding device has an arrangement surface in which a plurality of containers each being the container are two-dimensionally disposed, and
the moving device is configured to move at least one of the holding device and the rotating member relative to the other in a two-dimensional manner along the arrangement surface.

7. The opening apparatus for a container according to claim 1, further comprising an irradiation device that irradiates at least one of the first and second projections of the rotating member partially or entirely with ultraviolet ray.

8. The opening apparatus for a container according to claim 7, wherein the control device controls the irradiation device so that, after at least one of the first and second projections is brought into contact with the lid of the container with an analyte contained therein, the at least one of the projections brought into contact with the lid is partially or entirely irradiated with ultraviolet ray.

9. The opening apparatus for a container according to claim 1, wherein
the holding device has a function to heat the container, and
the control device controls
the opening device and the moving device so that the lid is opened at a first angle when the lid is brought to an open state before the holding device heats the container, and
the opening device and the moving device so that the lid is opened at a second angle when the lid is brought to an open state after the holding device heats the container, the second angle being larger than the first angle.

10. An analysis device comprising:
an opening apparatus according to claim 1; and
a dispensing device that dispenses a reagent into a container opened by the opening apparatus.

11. The analysis device according to claim 10, further comprising a pipette tip used to dispense liquid into the container, wherein
the dispensing device includes a nozzle that allows the pipette tip to be detachable therefrom, and
the control device controls the moving device so that when liquid is dispensed into the container via the pipette tip attached to the nozzle the pipette tip moves horizontally from a side of a tip of the lid toward the hinge.

12. The analysis device according to claim 1, wherein a polymerase chain reaction is used to analyze a gene.

13. A closing apparatus for a container including a container body, a lid, and a hinge that interconnects the container body and the lid, comprising:
a holding device that holds the container body;
a closing device including a rotating member and a drive device that rotates the rotating member, the rotating member being configured to apply a force to the lid in a direction to close the lid by rotating in contact with the lid in an open state; and
a control device that controls the closing device, wherein
when the lid in the open state is brought to a closed state, the control device performs a first closing control to apply a force to the lid in a direction to close the lid by rotating the rotating member so that a portion of the rotating member approaches the hinge while the portion of the rotating member is in contact with the lid.

14. The closing apparatus for a container according to claim 13, further comprising a moving device that moves at least one of the holding device and the rotating member relatively to the other, wherein
after the first closing control is executed, the control device executes a second closing control to control at least one of the closing device and the moving device so that the rotating member applies a force to the lid in a direction to close the lid while the rotating member is partially in contact with the lid.

15. A reagent kit comprising:
a plurality of containers; and
a packaging material that packages the plurality of containers,
the plurality of containers each including:
a container body, and
a lid connected to the container body via a hinge and capable of opening and closing with respect to the container body,
the plurality of containers being provided such that they are one-dimensionally disposed, with adjacent ones thereof connected to each other, the lid closed, and a reagent introduced in the container body.
